(19) United States
(12) Patent Application Publication
(10) Pub. No.: US 2025/0055000 A1
(43) Pub. Date: Feb. 13, 2025

(54) PHENAZINE DERIVATIVE-BASED ALKALINE BATTERY

(71) Applicant: City University of Hong Kong, Hong Kong (HK)

(72) Inventors: Chunyi ZHI, Hong Kong (HK); Huilin CUI, Hong Kong (HK); Jiaxiong ZHU, Hong Kong (HK)

(21) Appl. No.: 18/366,641

(22) Filed: Aug. 7, 2023

(51) Int. Cl. H01M 6/04; C07D 241/46; H01M 4/32; H01M 4/60; H01M 4/62; H01M 4/66

(52) U.S. Cl. CPC H01M 6/045; C07D 241/46; H01M 4/32; H01M 4/60; H01M 4/623; H01M 4/625; H01M 4/663; H01M 2004/027; H01M 2004/028

(57) ABSTRACT

The present invention relates to a phenazine derivative-based alkaline battery, which includes an anode formed by a phenazine derivative having 1 to 4 side group substituents, a cathode, a separator placed between the cathode and the anode and an electrolyte disposed in a space between the cathode and the anode. The present invention not only investigates the impact of hydroxyl substituents on electrochemical potential and reaction kinetics but also paves the way for the development of stable anodes for alkaline-based batteries. The phenazine derivative-based alkaline battery of the present invention exhibits a reversible capacity of at least 170 mAh g⁻¹ at 0.2 A g⁻¹, a power density of at least 20 KW kg⁻¹ at 10 A g⁻¹, a, and a stable cyclability over 9000 cycles.

### FIELD OF THE INVENTION

The present invention generally relates to the field of electrochemistry or energy storage. More specifically, the present invention provides a phenazine derivative-based alkaline battery.

### BACKGROUND OF THE INVENTION

Efforts are being made to address the critical challenges of fossil fuel depletion and environmental degradation by focusing on the development of renewable energy sources. Electrochemical energy storage technologies, such as organic lithium-ion batteries (LIBs), nickel-metal hydride (MH) batteries, and lead-acid batteries, are recognized as highly efficient energy storage devices for renewable energy^{1,2}.

Among these technologies, high energy density organic lithium-ion batteries (LIBs) have revolutionized the field of portable electronics and electric vehicles³. However, the occurrence of fires and explosions associated with LIBs has posed significant safety concerns, thereby driving the development of safe aqueous batteries for specific applications⁴.

Alkaline aqueous batteries (ANAB) are intrinsically safe and possess potential high voltages^{5,6,7}. However, corrosion caused by the side reactions under alkaline conditions is unavoidable for anode materials, which leads to the poor cycling stability of ANAB⁸. Cadmium metal is the first-generation anode material for alkaline aqueous batteries due to its high energy density, but was gradually phased out in ANAB due to its toxicity and strong “memory” effect. Subsequently, efforts were made to explore and utilize super-stable alloys as replacements for cadmium metals in ANAB⁹. Nevertheless, the low nominal battery voltage (1.2 V) and the high cost of the alloy are still unsatisfactory.

In recent years, zinc (Zn) metal has been extensively studied as an ideal anode material for alkaline-based batteries due to its low redox potential (−1.24V versus standard hydrogen electrode (SHE)) and remarkably high theoretical specific capacity (820 mAh·g⁻¹)^{4, 10, 11}. However, the relatively short cycle life caused by zinc oxidation and the formation of the undesirable Zn(OH)₄ passivation layer has hindered the development of Zn as an anode material in alkaline conditions^{2,8,12}.

To date, few metal materials can operate steadily in alkaline systems. In order to overcome these obstacles and achieve sustainable applications of alkaline-based batteries, it is crucial to design high-performance, low-cost, and environmental-friendly anode materials for ABAN. In addition to metals, efforts to design promising anode materials for ABANs have been extended to organic molecule materials. Organic molecules offer advantages such as low toxicity, cost-effectiveness, and structural diversity. Meanwhile, replacing metal anodes with organic electrode materials can potentially address issues such as dendrites, passivation, and corrosion associated with metal anodes.

Conjugated quinone polymers are extensively studied organic electrode materials for ABANs, which store charge through an “ion coordination” mechanism. These materials have demonstrated broad applicability in various electrolyte systems, including Al³⁺, Zn²⁺, Li⁺, H⁺, and NH₄^{+ 13-16}. However, the potentials of these materials are much higher than that of the zinc anode, resulting in low battery voltage and energy density.

Phenazine (PZ) derivatives have shown great promise as electrode materials due to their potential for high cycling stability^{17, 18}. The highly electronegative nitrogen atom in phenazine compounds significantly affects the density distribution of electron clouds in the ring, resulting in the transfer of electron density from the π system to the nitrogen atom. Compared to quinone compounds, phenazine derivatives with higher electron cloud density exhibit a greater reluctance to accept electrons, resulting in a relatively low redox potential¹⁹⁻²¹.

However, despite the lower redox potentials of phenazine derivatives compared to most organic compounds, they are still significantly more positive than that of zinc metal. In addition, it is also necessary to investigate the effects of grafted functional groups on the electrochemical redox properties of phenazine derivatives.

### SUMMARY OF THE INVENTION

Accordingly, the present invention aims to address the challenges associated with alkaline-based batteries. Specifically, the objective of the present invention is to develop a high-performance, low-cost, and environmentally-friendly anode material for alkaline rechargeable batteries, which can overcome issues such as short cycle life, zinc oxidation, and the formation of undesirable passivation layers. Additionally, the effects of grafted functional groups on the electrochemical properties of phenazine derivatives are also investigated in this invention.

In a first aspect, the present invention provides a phenazine derivative-based alkaline battery, which includes an anode formed by a phenazine derivative having 1 to 4 side group substituents, a cathode, a separator placed between the cathode and the anode and an electrolyte disposed in a space between the cathode and the anode. the phenazine derivative-based alkaline battery exhibits a reversible capacity of at least 170 mAh g⁻¹ at 0.2 A g⁻¹, a power density of at least 20 KW kg⁻¹ at 10 A g⁻¹, a, and a stable cyclability over 9000 cycles.

In accordance with one embodiment, the side group substituents include electron-donating groups or electron-withdrawing groups. The electron-donating groups include amino, hydroxyl, or a combination thereof, or the electron-withdrawing groups include methyl, carboxyl group, or a combination thereof.

In accordance with one embodiment, the anode and cathode include a current collector, at least one active material, one or more electronic conductive particles, and at least one binder.

In accordance with one embodiment, the current collector includes carbon nanotube paper, carbon cloth, and nickel foil.

In accordance with one embodiment, the at least one active material includes phenazine (PZ), 2-hydroxyphenazine (PZ-OH), and 1,2-dihydroxyphenazine (PZ-2OH).

In accordance with one embodiment, the one or more electronic conductive particles include carbon nanotubes, ketjenblack, and super P.

In accordance with one embodiment, the at least one binder includes sodium carboxymethylcellulose (CMC-Na), polyvinylidene difluoride (PVDF), and Polytetrafluoroetylene (PTFE).

In accordance with one embodiment, the cathode includes Ni(OH)₂ and Pt/C.

In accordance with one embodiment, the electrolyte includes a solvent and a solute, and the solvent includes deionized water, the solute includes sodium hydroxide, potassium hydroxide, and lithium hydroxide. The concentration of the solute is in a range of 1 M to 6 M.

Optionally, the phenazine derivative-based alkaline battery further includes a graphene oxide (GO) film, the phenazine derivative-based alkaline battery is assembled by inserting the GO film between the anode and the separator, and the electrolyte is added between the anode and the cathode. The GO film is formed by the following steps: mixing an ethanol solvent and GO with a mass fraction of 2% to 3% to obtained a suspension, filtering and drying the suspension at 70° C. for 24 hours. Finally, the free-standing GO film is obtained after peeling it off from the filter paper.

In accordance with one embodiment, the phenazine derivative-based alkaline battery retains at least 10% of the initial capacity, and the discharge potential of the phenazine derivative-based alkaline battery is lower than −0.8 V.

In accordance with another embodiment, an increase in the number of the side group substituents results in a reduction in electron affinity and a decrease in the redox potential.

In a second aspect, the present invention provides a method for preparing the phenazine derivative, including preparing at least one precursor including at least one benzene derivative, at least one phenylenediamine derivative, and a solvent; dissolving the at least one benzene derivative into the solvent to form a uniform solution A; mixing the at least one phenylenediamine derivative with the solution A to form a suspension; and filtering and washing the suspension with deionized water until a filtrate becomes colorless. The at least one benzene derivative and the at least one phenylenediamine derivative has a molar ratio in a range of 1:1-1:0.8.

In accordance with one embodiment, the method further contains concentrating the mixture at reduced pressure and adding deionized water to obtain a suspension before filtering and washing the suspension.

In accordance with one embodiment, the at least one benzene derivative includes benzoquinone or 2,5-dihydroxy-1,4-benzoquinone.

In accordance with one embodiment, the at least one phenylenediamine derivative has the following chemical structure:

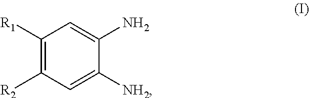

wherein the R₁ and R₂ are independently selected from H, Cl, COOH, or CH₃.

In accordance with one embodiment, the solvent includes ethanol, deionized water, or a combination thereof.

In accordance with another embodiment, the step of mixing the at least one phenylenediamine derivative with the solution A to form a suspension has a reaction temperature ranges from −10° C. to 100° C., and a reaction time ranges from 5-10 hours.

The present invention pertains to an economical and environmentally friendly synthetic method for producing a range of side-group substituted phenazine organic materials. The electrochemical potential of these organic materials can be customized by altering the type and quantity of side group substitutions. By incorporating electron-donating groups such as amino and hydroxyl groups, the potential of phenazine derivatives can be reduced, enabling the development of ultra-low voltage, highly stable organic materials for alkaline electrochemical storage devices.

Compared to existing technologies, the present invention offers the following major advantages:
(1) The preparation method for the phenazine derivatives is simple, with low-cost raw materials, and the reaction process is environmentally friendly, making it conducive to large-scale production.
(2) The redox potential of the prepared phenazine derivatives can be adjusted by modifying the type and amount of side functional groups, opening up possibilities for the application of phenazine derivatives in the field of energy.
(3) The phenazine derivatives with hydrophilic side groups exhibited superior rate capability, attributed to the formation of hydrogen bonds between hydroxyl groups and water molecules. These hydrogen bonds facilitate the rapid transport of H+ within the material through Grotthuss mechanisms.
(4) The phenazine derivative exhibits ultra-low redox potential and ultra-high cycle stability in various alkaline battery systems, including alkaline nickel-based systems and alkaline air battery systems.

### BRIEF DESCRIPTION OF THE DRAWINGS

Embodiments of the present invention are described in more detail hereinafter with reference to the drawings, in which:

FIG. 1A shows molecule structure and calculated corresponding ESP distribution of three phenazine derivatives. FIG. 1B shows ¹H NMR spectra of three phenazine derivatives. FIG. 1C shows the FT-IR spectra of three phenazine derivatives. FIG. 1D shows the CV curves of three phenazine derivatives. FIG. 1E shows galvanostatic discharge/charge curves of three phenazine derivatives at a current density of 1 A g⁻¹. FIG. 1F shows calculated frontier molecular orbital energy and electron affinity of the three molecules. FIG. 1G shows a comparison of the discharge potential between PZ-2OH and reported organic anode in alkaline-based system;

FIG. 2A shows ¹H NMR spectrum of PZ-OH. FIG. 2B shows ¹H NMR of PZ-2OH;

FIG. 3A shows XRD patterns of the synthesized phenazine derivatives. FIG. 3B shows optical photographs of the as-synthesized phenazine derivatives. FIG. 3C shows scanning electron microscope (SEM) images of the as-synthesized phenazine derivatives;

FIG. 4 shows TGA curves of three phenazine derivatives under an N₂ atmosphere;

FIG. 5 shows optimized structure and calculated LUMO and HOMO energy levels of three phenazine derivatives;

FIG. 6A shows rate capabilities of three phenazine derivatives at various current densities. FIG. 6B shows a schematic diagram of battery impedance spectrum. FIG. 6C shows the EIS curve of PZ, PZ-OH, and PZ-2OH molecules. FIG. 6D depicts the relationship between the real part of the impedance and ω^{1/2}. FIG. 6E shows the Arrhenius plots of ln (R_{ct}) vs. 1000/T for PZ, PZ-OH, and PZ-2OH molecules. FIG. 6F shows the schematic illustration for the ion conduction manner in the hydrogen bonding network in PZ-2OH. FIG. 6G shows plots of calculated RDG isosurface vs. sign (λ₂) ρ for PZ-2OH;

FIG. 7 shows Nyquist plots at various temperatures of PZ-2OH;

FIG. 8 shows the CV curves at different scan rates and the corresponding log iₚₑₐₖ versus log v of PZ, PZ-OH and PZ-2OH;

FIG. 9 shows the capacitive contributions of three phenazine derivatives at different scan rates;

FIG. 10 shows the coefficient b of PZ, PZ-OH, and PZ-2OH;

FIG. 11 shows SEM images of GO membranes;

FIG. 12A shows the CV profile of GO∥Ni(OH)₂ battery at 0.5 V s⁻¹. FIG. 12B shows the GCD profile of GO∥Ni(OH)₂ battery at 0.2 A g⁻¹;

FIG. 13A shows CV curves of the PZ-2OH∥Ni(OH)₂ battery. FIG. 13B shows rate capability and coulombic efficiency at different current density. FIG. 13C shows the comparison of the energy density and power density of the PZ-2OH∥Ni(OH)₂ battery with other energy storage systems. FIG. 13D shows the comparison of the battery discharge voltages of organic electrode material in acid electrolytes, mild electrolytes, and alkaline electrolytes. FIG. 13E shows GCD curves of PZ-2OH∥Ni(OH)₂ full cell at the temperature range of −30° C. to 20° C. and current density of 1 A g⁻¹. FIG. 13F shows the cycle life of PZ-2OH∥Ni(OH)₂ battery at a current density of 0.5 A g⁻¹. FIG. 13G shows comprehensive performance evaluation, including the price, energy density, voltage, toxicity and stability, between the conventional Ni-based batteries (Zn∥Ni, MH∥Ni, Cd∥Ni) and PZ-2OH∥Ni(OH)₂;

FIG. 14 shows a price comparison of the prepared phenazine derivatives with other anode materials for alkaline nickel-based batteries;

FIG. 15A shows GCD curves of PZ-2OH∥Ni(OH)₂ at different current density. FIG. 15B shows CV curve of PZ-2OH∥Ni(OH)₂ full cell at different scan rates;

FIG. 16 shows the linear fitting of the peak current density with different scan rates of the PZ-2OH∥Ni(OH)₂ full cell;

FIG. 17A shows the charge/discharge curve of the PZ-2OH anode at the current density of 0.5 A g⁻¹. FIG. 17B shows full spectra of PZ-2OH electrodes at selected points. FIG. 17C shows high resolution N 1s spectra of PZ-2OH electrodes at selected points. FIG. 17D shows In-situ Raman spectra of PZ-2OH during the process of charge and discharge. FIG. 17E shows typical Raman spectra of PZ-2OH electrodes at annotated points. FIG. 17F shows Ex situ FTIR of PZ-2OH electrodes after different cycles;

FIG. 18 shows a scheme of the redox reaction mechanism of PZ-2OH anode;

FIG. 19A shows typical GCD curves of the PZ-2OH∥Air battery. FIG. 19B shows rate performance of the PZ-2OH∥Air battery at a range of current densities. FIG. 19C shows the cycling stability of the PZ-2OH∥Air battery; and

FIG. 20 shows CV curves of PZ-2OH∥Air battery at scan rate of 5 mV s⁻¹.

### DETAILED DESCRIPTION

The present invention will be described in detail through the following embodiments with appending drawings. It should be understood that the specific embodiments are provided for an illustrative purpose only, and should not be interpreted in a limiting manner. Those skilled in the art will appreciate that the invention described herein is susceptible to variations and modifications other than those specifically described.

The invention includes all such variation and modifications. The invention also includes all of the steps and features referred to or indicated in the specification, individually or collectively, and any and all combinations or any two or more of the steps or features. Other aspects and advantages of the invention will be apparent to those skilled in the art from a review of the ensuing description.

Alkaline aqueous batteries are inherently safe and have the potential for high voltages. In particular, the alkaline-based aqueous batteries have garnered significant attention in research due to their high voltage, low cost, and excellent safety features. However, traditional metal anode materials in alkaline conditions are prone to corrosion, resulting in poor cycling stability of alkaline aqueous batteries. In addition, the traditional metal anodes used in alkaline electrolytes often exhibit poor stability and suffer from severe side reactions.

Organic materials present a promising solution to overcome these challenges, which offer potential solutions to issues such as dendrite formation, passivation, and corrosion of metal anodes. This is because organic materials consist of non-metallic elements (such as C, H, O, N, and S), unlike inorganic materials. However, they typically lack sufficient negative potential to be used as anodes.

Accordingly, the present invention provides a phenazine derivative-based alkaline battery, which includes an anode formed by a phenazine derivative having 1 to 4 side group substituents, a cathode, a separator placed between the cathode and the anode and an electrolyte disposed in a space between the cathode and the anode. the phenazine derivative-based alkaline battery exhibits a reversible capacity of at least 170mAh g⁻¹ at 0.2 A g⁻¹, a power density of at least 20 KW kg⁻¹ at 10 A g⁻¹, a, and a stable cyclability over 9000 cycles.

Phenazine (PZ) derivatives are highly promising electrode materials due to their potential for high cycling stability. Although the redox potentials of phenazine derivatives are lower than those of most organic compounds, they are still significantly more positive than zinc metal. It has been discovered that the introduction of hydroxyl groups can effectively reduce the redox potential of the organic material by approximately 0.4V. Additionally, the formation of fast ion transport channels through intramolecular hydrogen bonds greatly enhances the redox kinetics of the material.

In one of the embodiments, the side group substituents may be electron-donating groups or electron-withdrawing groups. The electron-donating groups include, but are not limited to, amino, hydroxyl, or a combination thereof, or the electron-withdrawing groups include, but are not limited to, methyl, carboxyl group, or a combination thereof.

In one of the embodiments, the anode materials of the phenazine derivative-based alkaline battery are phenazine or hydroxy-substituted phenazine, which may include, but are not limited to, phenazine (PZ), 2-hydroxyphenazine (PZ-OH), and 1,2-dihydroxyphenazine (PZ-2OH).

These phenazine derivatives have different numbers of hydroxyl group. The comprehensive studies demonstrate that the hydroxyl-substituted groups adjacent to the phenazine can lower the redox potential by reducing the ability to lose electrons and improve the rate performance by forming hydrogen bonds with water molecules in the electrolyte. Meanwhile, the phenazine derivatives with hydroxyl group provided a fast charge transport channel through intermolecular interactions of hydroxyl functional group, that reduced the charge-transfer impedance and delivered superior rate performance compared with the phenazine.

In one of the embodiments, the cathode materials may be Ni(OH)₂ or Pt/C.

Preferably, alkaline battery of the present invention may be PZ∥Ni(OH)₂ battery, PZ-OH∥Ni(OH)₂ battery, or PZ-2OH∥Ni(OH)₂ battery.

In one of the embodiments, the introduction of hydroxyl to phenazine acted as an electron donor group can remarkably lower the discharge potential. For example, the discharge potential is decreased from −0.78 V (PZ) to −1.07 V (PZ-2OH).

In one of the embodiments, the anode and cathode may include a current collector, at least one active material, one or more electronic conductive particles, and at least one binder. The current collector includes, but is not limited to, carbon nanotube paper, carbon cloth, and nickel foil. The at least one active material includes, but is not limited to, PZ, PZ-OH, and PZ-2OH. The one or more electronic conductive particles include, but are not limited to, carbon nanotubes, ketjenblack, and super P. The at least one binder includes, but is not limited to, CMC-Na, PTFE, and PVDF.

In one of the embodiments, the electrolyte includes a solvent and a solute, and the solvent may be deionized water, the solute may be sodium hydroxide, potassium hydroxide, and lithium hydroxide. The concentration of the solute is in a range of 1 M to 6 M.

Optionally, the phenazine derivative-based alkaline battery further includes a graphene oxide (GO) film, the phenazine derivative-based alkaline battery is assembled by inserting the GO film between the anode and the separator, and the electrolyte is added between the anode and the cathode.

In one of the embodiments, the invention can improve the battery capacity and energy density of alkaline-based batteries. For example, the phenazine derivative-based alkaline batteries exhibit a high reversible capacity of at least 170 mAh g⁻¹ at 0.2 A g⁻¹, high power density of at least 20 KW kg⁻¹ at 10 A g⁻¹, superior stable cyclability over 9000 cycles.

More preferably, the optimized PZ-2OH∥Ni(OH)₂ batteries exhibit a high capacity of 208 mAh g⁻¹ (anode) at 0.2 A g⁻¹, a high energy density of 247 Wh kg⁻¹ (anode), high power density of 26.2 KW kg⁻¹ at 10 A g⁻¹, and exceptional cycling stability with over 9000 cycles and a low capacity decay rate of approximately 0.075% per cycle.

In one of the embodiments, the discharge potential of the phenazine derivative-based alkaline battery is lower than −0.8 V. For example, the discharge potential can be −0.85 V, −0.9 V, −0.95 V, or −1.00 V, etc.

In one of the embodiments, the phenazine derivative-based alkaline batteries retain at least 10% of the initial capacity. For example, the initial capacity was retained by at least 11%, at least 12%, at least 13%, at least 14%, at least 15%, at least 16%, at least 17%, at least 18%, at least 19%, or at least 20%.

Preferably, the phenazine derivative-based alkaline batteries retain 20% of the initial capacity.

In one of the embodiments, the present invention also demonstrates the feasibility of utilizing PZ-2OH in an alkaline PZ-2OH∥Air battery, thus further confirming its applicability under alkaline conditions.

In another aspect, the present invention provides a method for preparing the phenazine derivative, including preparing at least one precursor including at least one benzene derivative, at least one phenylenediamine derivative, and a solvent; dissolving the at least one benzene derivative into the solvent to form a uniform solution A; mixing the at least one phenylenediamine derivative with the solution A to form a suspension; and filtering and washing the suspension with deionized water until a filtrate becomes colorless. The at least one benzene derivative and the at least one phenylenediamine derivative has a molar ratio in a range of 1:1-1:0.8.

In one of the embodiments, the at least one benzene derivative includes, but is not limited to, benzoquinone or 2,5-dihydroxy-1,4-benzoquinone.

In one of the embodiments, the at least one phenylenediamine derivative has the following chemical structure:

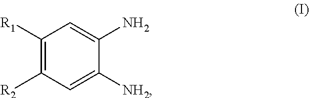

wherein the R₁ and R₂ are independently selected from H, Cl, COOH, or CH₃.

In one of the embodiments, the solvent may be ethanol, deionized water, or a combination thereof.

The following examples illustrate the present invention and are not intended to limit the same.

### EXAMPLE

### Example 1—Materials and Methods

### Material

All chemicals were purchased from commercial suppliers and used without any further purification, including phenazine (PZ, Aldrich), benzoquinone (Aldrich), 1,2-phenylenediamine (Aldrich) and 2,5-dihydroxy-1,4-benzoquinone (Aldrich), PTFE (Macklin), isopropyl alcohol (Macklin), potassium hydroxide (KOH, Aldrich), Lithium hydroxide (LiOH, Macklin), ketjenblack (KB, ECP-600JD, Lion Corporation), isopropanol alcohol (Aldrich), Pt/C (20 wt. % in Pt, Macklin), (Methyl sulfoxide)-d₆ (DMSO-d₆, Aldrich), Nafion D-520 dispersion (5% w/w in water and 1-prppanol, Alfa Aesar) and graphite oxide aqueous dispersion (GO, 10 mg/g, GaoxiTech). Phenazine derivatives of 2-hydroxyphenazine (PZ-OH) and 1,2-dihydroxyphenazine (PZ-2OH) were prepared according to the following steps.

### Electrochemical Measurement Methods for Batteries

The cyclic performance, electrochemical properties of batteries were characterized by the LAND CT2001A device and electrochemical workstation CHI 760D at 25° C. The above electrochemical properties were measured at 25° C. The low-temperature performance of PZ-2OH∥Ni(OH)₂ battery was tested in a cryogenic box (−30˜20° C.). All current densities, specific capacities, and energy densities in this work were calculated based on the mass of phenazine derivatives anode material. All the batteries were activated in the initial three cycles at the current density of 0.2 A g⁻¹. For each electrochemical test, at least three-coin cells were assembled for test.

### Calculation of the Energy Density and Power Density

The energy density E (W h kg⁻¹) and power density P (W kg⁻¹) values of the

battery were calculated as follows:

$$E = \frac{\int IV ( t ) dt}{3.6 m} ; \qquad ( 1 )$$

$$P = \frac{3600 E}{t} , \qquad ( 2 )$$

where the I is the applied current (A), V is the potential of device (V), t is the discharge time(s), and m is the weight of electrode (g).

### Calculation of the Capacitive Contribution

The ion and electron transport kinetics and the capacitive contribution were calculated according to the following equation:

$$i = {kv}^{b} , \qquad ( 4 )$$

where k and b are constants. The above equation can be rearranged as follow:

log (i)=log(k)+b*log(v) (5),

by linear plotting log (i) versus log (v), the value of b can be obtained.

### Example 2—Preparation of 2-hydroxyphenazine (PZ-OH) and 1,2-dihydroxyphenazine (PZ-2OH)

Three types of phenazine derivatives (PZ, PZ-OH, PZ-2OH) were successfully synthesized using a simple and environmentally friendly condensation method. The precursors used for the synthesis were low-cost diaminobenzene and quinone.

### Synthesis of PZ-OH

First, 1.08 g of benzoquinone (10 mmol) was added into a 100 mL anhydrous alcohol solution, forming solution A. Then, 1.08 g of 1,2-phenylenediamine (10 mmol) was dropped into solution A at −10° C. and continuously stirred for 2 h. Subsequently, the obtained mixture was concentrated to 10 mL at reduced pressure, and then 50 mL of deionized water was poured into the concentrated mixture. The resulting suspension was filtered and washed with deionized water until the filtrate became colorless. The yield of PZ-OH was approximately 70%.

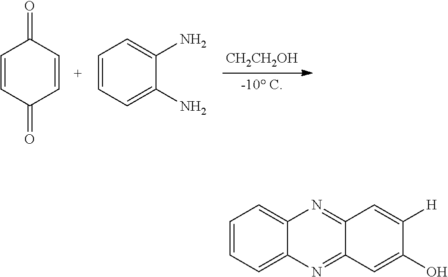

### Synthesis of PZ-2OH

First, 0.71 g of 2,5-dihydroxy-1,4-benzoquinone (0.51 mmol) and 130 mL DI water were mixed in a round bottomed flask under a nitrogen atmosphere, forming solution A at 80° C. Then, 0.5 g of 1,2-phenylenediamine (0.46 mmol) was dropped into solution A and continuously stirred for 7 h. Subsequently, the mixture was naturally cooled to room temperature and stirred overnight. The resulting suspension was filtered and washed with deionized water until the filtrate became colorless. The yield of PZ-OH was approximately 80%.

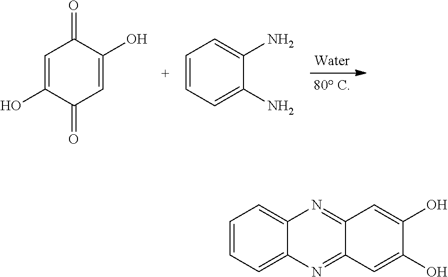 [SMILES: O=C1C=C(O)C(=O)C=C1O ; Nc1ccccc1N ; "Water" ; Oc1cc2nc3ccccc3nc2cc1O]

### Example 3—Characterization of synthesized PZ, PZ-OH and PZ-2OH

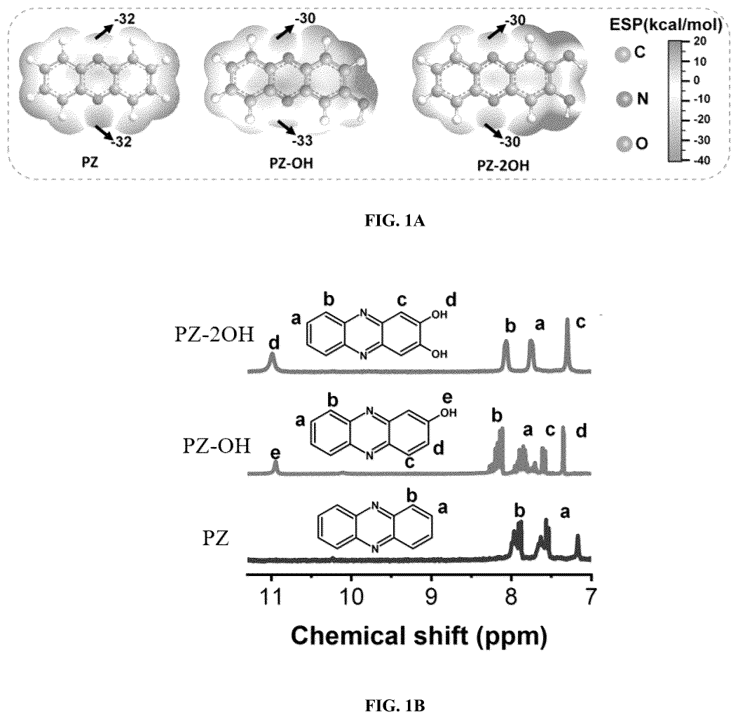

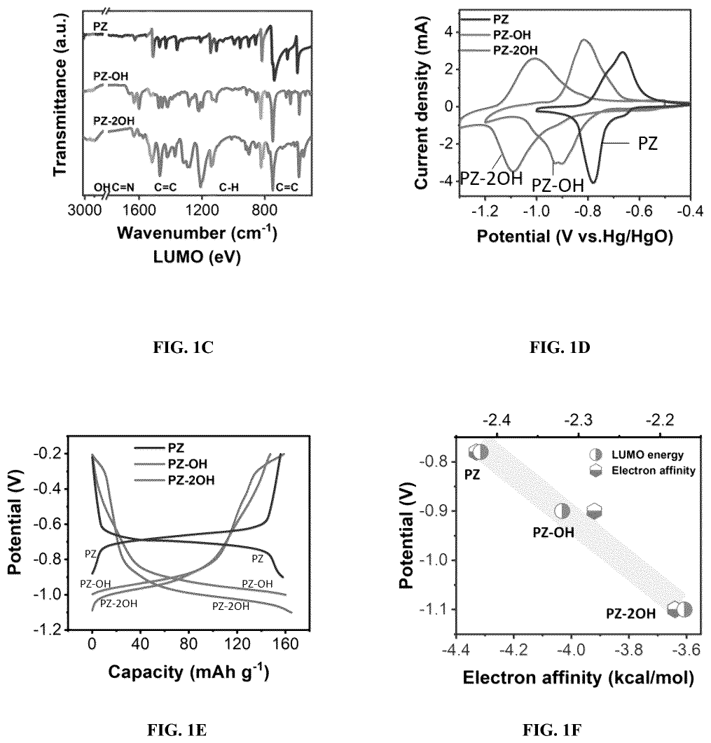

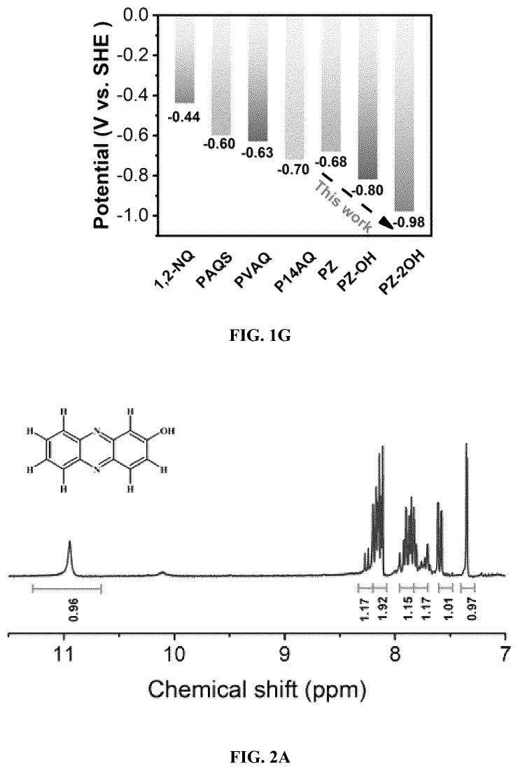

The chemical structures of these phenazine derivatives were shown in FIG. 1A and were confirmed by 1H nuclear magnetic resonance (NMR) and Fourier transform infrared spectroscopy (FT-IR). The proton nuclear magnetic resonance (1H NMR) was collected using Nuclear Magnetic Resonance 300 (NMR 300). The Fourier-Transform Infrared Spectroscopy (FT-IR) was obtained on a PerkinElmer Spectrum II spectrometer to characterize the changes of the specific chemical groups.

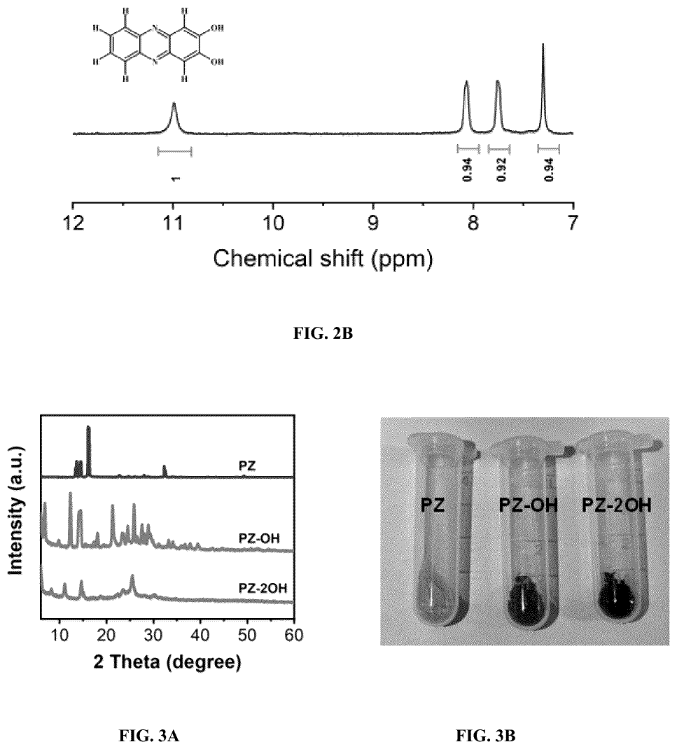

Referring to FIG. 1B, the 1H NMR spectra showed significant chemical shifts for PZ-OH (300 MHz, DMSO-d₆) at 10.95 (s, 1H), 8.19 (dd, J=8.7, 1.5 Hz, 1H), 8.13 (dd, 1H), 7.90 (ddd, J=8.6, 6.6, 1.7 Hz, 1H), 7.83 (ddd, J=8.2, 6.6, 1.6 Hz, 1H), 7.60 (dd, J=9.4, 2.6 Hz, 1H), 7.35 ppm (d, J=2.6 Hz, 1H) (FIG. 2A); and for and PZ-2OH (300 MHz, DMSO-d₆) at 10.99 (s, 2H), 8.07 (s, 2H), 7.74 (s, 2H), 7.30 ppm (s, 2H) (FIG. 2B). This indicated that PZ-OH and PZ-2OH were successfully synthesized.

The synthetic structure was further characterized by FT-IR (FIG. 1C). The emerging absorption peaks at 1627 cm⁻¹, 1518 cm⁻¹, 1145 cm⁻¹, and 827 cm⁻¹ in the FTIR spectra of phenazine derivatives indicated the formation of the C═N, C═C, and C—H linkages, while the broad FTIR spectra of PZ—OH and PZ-2OH at approximately 3500 cm⁻¹ evidence the existence of hydroxyl group.

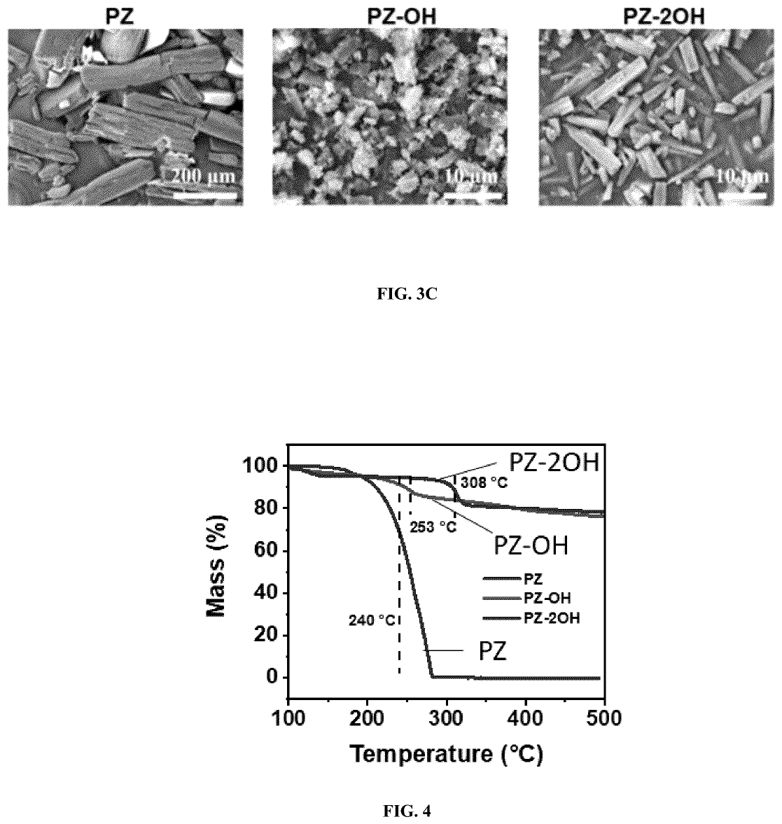

Turning to FIG. 3A, the powder X-ray diffraction patterns (XRD) were also tested. X-ray diffractometer equipment (XRD; Bruker, D2 Avance) was used to record the XRD pattern. The results demonstrated that phenazine-based organics had relatively ordered-aggregated structures due to the π-π stacking of individual phenazine derivative-based molecules. Meanwhile, the prepared phenazine derivatives exhibit different bulk colors, ranging from yellow for PZ, to brown for PZ-OH, and finally to gray for PZ-2OH (FIG. 3B).

In addition, the morphology and microstructure were observed by scanning electron microscope (SEM; SU4800, Hitachi). The SEM images showed that PZ had nanorod morphology with a length of approximately 300 μm, PZ-OH has nanocluster morphology with a size of approximately 10 μm, and PZ-2OH has nanorod morphology with a size of approximately 3 μm (FIG. 3C).

Thermogravimetric Analysis (TGA) was carried out on TG/DTA 6300 to obtain the thermodynamic stability of materials, and the heating rate was 5° C. min⁻¹ from room temperature to 500° C. under N₂. Referring to FIG. 4, the decomposition temperature gradually increased from 240° C. (PZ) to 253° C. (PZ-OH) and 308° C. (PZ-2OH) with increasing number of hydroxyl groups, suggesting that hydroxyl introduction contributes to thermal stability of organic materials. These results demonstrate that the substituent groups have a significant impact on the physical properties and electronic structure of these phenazine derivatives.

For further analysis of the structure and functional sites, density functional theory (DFT) calculations, commonly used in organic chemistry to derive electrophilicity or nucleophilicity, were employed to simulate the molecular electrostatic potential (ESP). All DFT simulations were performed by the Gaussian 16 software package. The structures and the energy levels of the highest occupied molecular orbital (HOMO)/lowest unoccupied molecular orbital (LUMO) of phenazine derivatives were fully optimized at B3LYP/6-31+G (d, p) level. Molecular electrostatic potential (ESP) results were performed with Multiwfn 3.3.9 programs and the visualization of ESP plots was carried out by Visual Molecule Dynamics (VMD) software. The electron affinity was defined by the change of the free energy between the neutral system to the negatively charged system.

At the van der Waals surface of the phenazine derivatives molecule (FIG. 1A), the molecules with more negative ESP exhibited a tendency towards electrophilic reactions²². Results from the ESP mapping showed that the regions of the C═N bond in the three phenazine derivatives (PZ, PZ-OH, PZ-2OH) exhibited negative ESP values, indicating that these regions were identified as highly reactive sites.

### Example 4—Electrochemical Measurements

The CR2032 coin-type cells were assembled in an air atmosphere for the electrochemical measurements of phenazine derivatives. The anodes were prepared by compressing the mixture of the phenazine derivatives powder, ketjenblack (KB), and polytetrafluoroethylene (PTFE) binder (mass ratio: 6:2.5:1.5) and a certain amount of isopropyl alcohol solvent on the nickel foam, after drying at 60° C. overnight under vacuum, the anode with a mass loading of 1-1.5 mg cm⁻² was obtained. 200 μL aqueous electrolyte with 6 M KOH and 1 M LiOH was employed as the electrolyte.

Regarding the phenazine derivatives ∥Ni(OH)₂ battery, an excess amount of Ni(OH)₂ was used as the cathode, and the Ni(OH)₂ electrode was obtained from the commercial nickel-metal hydride (Ni-MH) battery that can eliminate the impact of the cathode material on the electrochemical performance of the full cell.

Regarding the organic ∥ Air battery, the Pt/C cathode was prepared by the following steps: blending 5 mg of Pt/C with 50 μL of Nafion solution (5 wt % Nafion in a solution of isopropanol:water=1:1 vol.). The mixture was sonicated for 30 minutes, and the resulting slurry was cast onto a clean carbon cloth. The cathode was then dried at 80° C. for 12 hours in a vacuum oven.

Regarding the full battery assembly, a free-standing graphene oxide (GO) film was inserted between the anode and the separator to prevent the dissolution of anode materials. The GO film was prepared using a simple filtration method. First, 1 g of GO aqueous solution was dispersed in 40 mL of ethanol and subjected to ultrasonic treatment for 60 minutes. The resulting suspension was then filtered and dried at 70° C. for 24 hours. Finally, the free-standing GO film with a mass of 0.6 mg cm⁻² was obtained after peeling it off from the filter paper.

In three electrode system, the phenazine derivative, Pt metal, and Hg/HgO were used as anodes, counter electrodes, and reference electrodes, respectively.

The electrochemical performances of the as-synthesized PZ, PZ-OH and PZ-2OH were evaluated in 1 M KOH aqueous alkaline electrolyte. Cyclic voltammetry (CV) and Electrochemical Impedance Spectroscopy (EIS) curves were carried out on an electrochemical workstation (CHI 760E). Referring to FIG. 1D, CV curves of the three phenazine derivatives at 5 mV s-1 scan rate showed that there was a significant decrease in the oxidation and reduction potential upon introduction of hydroxy groups. The reduction potential dropped dramatically from −0.78 V to −0.9 V and then down to −1.07 V for PZ, PZ-OH, and PZ-2OH, respectively. The galvanostatic charge/discharge (GCD) tests, rate capability, and cycle performance were measured on battery test systems (LAND CT2001A). The above results consistent with the GCD curves, which showed the average discharge voltage decreased from PZ, PZ-OH to PZ-2OH, respectively (FIG. 1E). However, the capacities of these three phenazine derivatives were not significantly changed, all being approximately 160 mAh g⁻¹ at a current density of 0.5 A g⁻¹.

To understand the potential characteristics, the electron affinities of three phenazine derivatives were analyzed using the first principle DFT method utilizing B3LYP. The electron affinity represents the free energy variation between the neutral system to the negatively charged system.

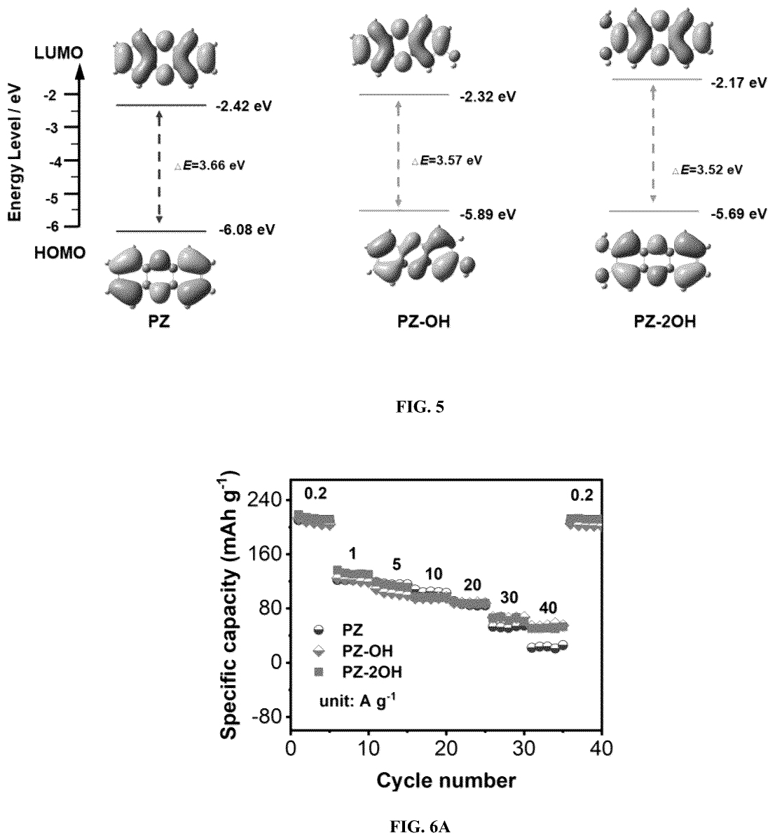

Referring to FIG. 1F, the redox potentials exhibited linear correlations with both the electron affinity and the LUMO energy levels. FIG. 1F showed that an increase in the number of hydroxyl groups generally leaded to a reduction in electron affinity and a decrease in the redox potential. The different redox potentials of three phenazine derivatives were interpreted using the LUMO energy levels (FIG. 5). The summary of the free energy of neutral and charged system and calculated electron affinity (EA) of PZ, PZ-OH and PZ-2OH was shown in Table 1. These results suggested that the high potential of molecular electrode materials could be predictable by more negative electron affinity and LUMO energy levels^{24, 25}.

TABLE 1

| | Free energy of | Free energy of | | |
|---|---|---|---|---|
| | neutral system | charged system | EA | EA |
| Material | (Hartree) | (Hartree) | (Hartree) | (kcal/mol) |
| PZ | −571.6190 | −572.8284 | −1.2094 | −4.34 |
| PZ-OH | −646.8415 | −648.0456 | −1.2041 | −3.96 |
| PZ-2OH | −722.0589 | −723.2564 | −1.1975 | −3.64 |

More importantly, the discharge potential of the PZ-2OH is lower than the cases of those reported organic anodes, such as 1,2-naphthoquinone (1,2-NQ), poly(anthraquinonyl sulfide) (PAQS), poly(2-vinylanthraquinone) (PVAQ), poly(1,4-anthraquinone) (P14AQ), in the aqueous alkaline electrolyte (FIG. 1G and Table 2).

TABLE 2

| Organic anode | Discharge potential (V vs. SHE) |
|---|---|
| 1,2-NQ | −0.44 |
| PAQS | −0.60 |
| PVAQ | −0.63 |
| P14AQ | −0.70 |
| PZ | −0.68 |
| PZ-OH | −0.80 |
| PZ-2OH | −0.98 |

### Example 5—Effects of the Number of Hydroxide Group on Electrochemical Performance

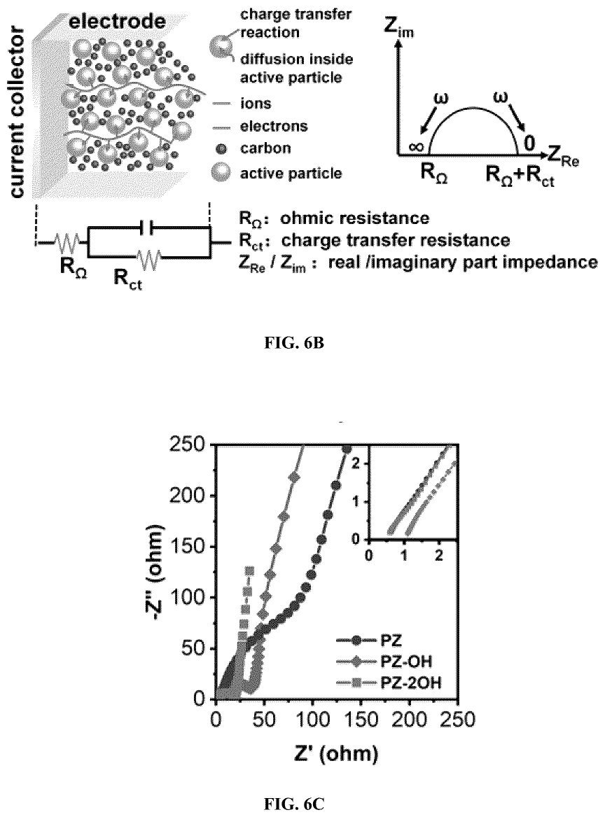

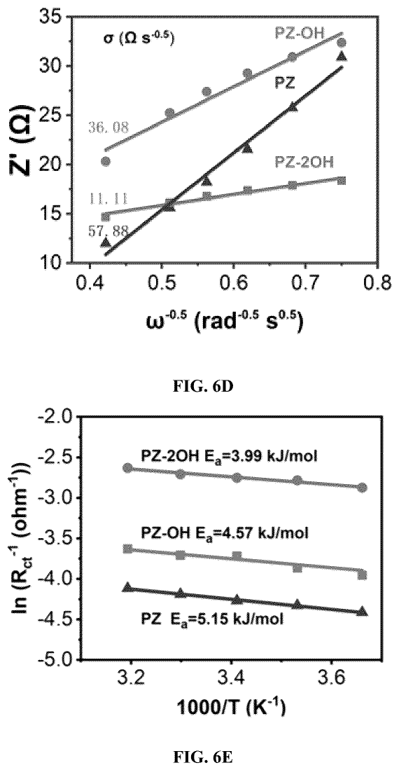

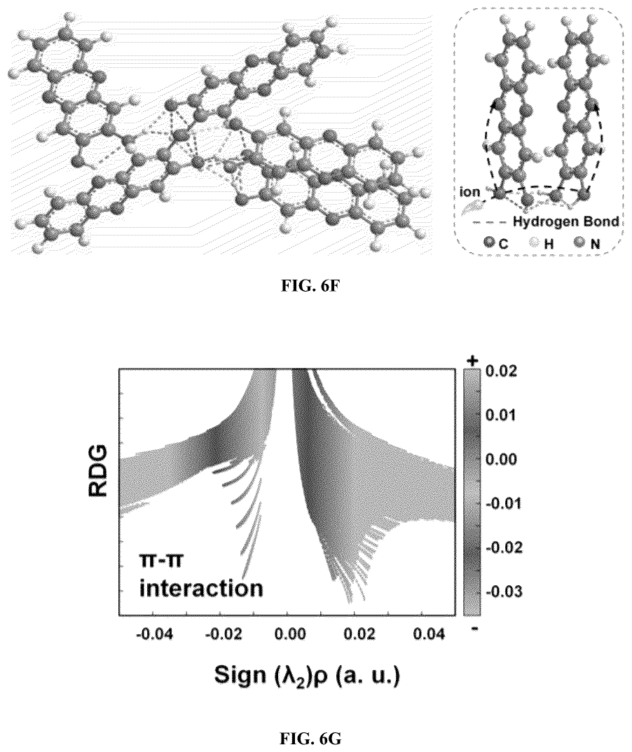

To investigate the influence of hydroxyl groups on electrochemical performance, the effects of hydroxyl on phenazine electrochemical properties were explored, which discloses a strong structure-electrochemical activity correlation. First, the rate capability of three phenazine derivatives was tested across a range of current densities from 0.2 mA g⁻¹ to 40 mA g⁻¹. Turning to FIG. 6A, the hydroxide-enriched PZ-OH and PZ-2OH exhibited better rate capability than the PZ, particularly with high current densities. The capacity of PZ-2OH and PZ-OH remained at 50 mAh g⁻¹ when the current density reaches 40 mA g¹, retaining 20% of the initial capacity, whereas the PZ only had a capacity of 20 mAh g⁻¹.

To further understand the effect of hydroxy-substituted phenazine on rate performance, electrochemical impedance spectroscopy (EIS) was employed for the analysis of the electrochemistry kinetics mechanism. Since the three electrodes were all performed in the same electrolytes, the impedance caused by the diffusion process could be ignored. The effect on rate capability was mainly attributed to the difference in the charge transfer process, which was controlled by the electrochemical reaction steps.

As shown in FIG. 6B, the electrochemical system could be simplified as the joint-controlled ohmic impedance and electrochemical transfer, which could be intuitively measured by the EIS of different electrodes. As demonstrated by the Nyquist plots, the ohmic impedance of the PZ-2OH was smaller than that of the PZ-OH and PZ (FIG. 6C), which was consistent with the Highest Occupied Molecular Orbital (HOMO)-LUMO energy level difference (PZ-2OH (ΔE=3.52 eV)<PZ-OH (ΔE=3.57 eV)<PZ (ΔE=3.66 eV)) (FIG. 5). The low HOMO-LUMO energy level gap indicated that electrons could easily transition from highest to lowest occupied orbit²³. Thus, the relatively high conductivity (smaller ohmic impedance) of PZ-2OH contributed to the high-rate capability.

In addition, the ion diffusion resistance (σ) was obtained by quantitatively analyzing the real part of impedance (Z′), which was linear with the reciprocal of the square root of frequency (ω^{-0.5})³². The smaller slope of the linear tendency stands for the faster ion transport. Referring to FIG. 6D, the PZ-2OH electrode delivered the lowest σ value of 11.11 Ωs^{-0.5}, followed by PZ-OH (36.08 Ωs^{-0.5}) and PZ (57.88 Ωs^{-0.5}), indicating that the PZ-2OH anode possessed a more rapid ion transport kinetics (lower charge-transfer impedance).

Moreover, the charge transfer behavior was further evaluated by the activation energy (Ea). The Eₐ was calculated according to the EIS spectra at different temperature, which can be calculated by the following equation:

1/R_{ct}=A₀ e^{-Ea/RT},

where R_{ct}, R and T presents the charge transfer resistance (Ω), gas constant (8.314 J mol⁻¹ K⁻¹), and the testing temperature (298 K), respectively.

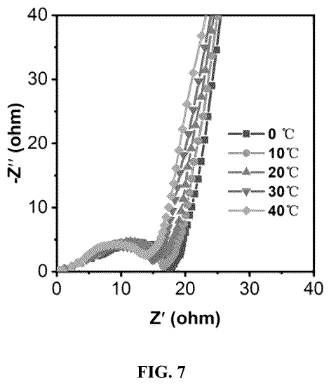

In accordance with the Arrhenius equation, the Ea value of PZ-2OH was 3.99 kJ mol⁻¹ (FIG. 6E and FIG. 7), which was smaller than PZ-OH (4.57 kJ mol⁻¹) and PZ (5.15 kJ mol⁻¹), demonstrating that the introduction of hydroxyl guaranteed fast ion diffusion and efficient charge transport.

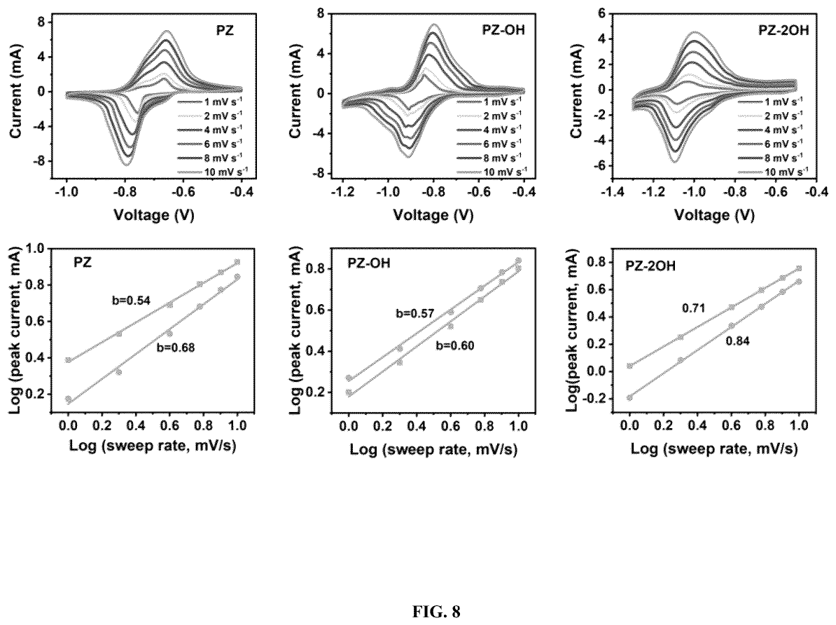

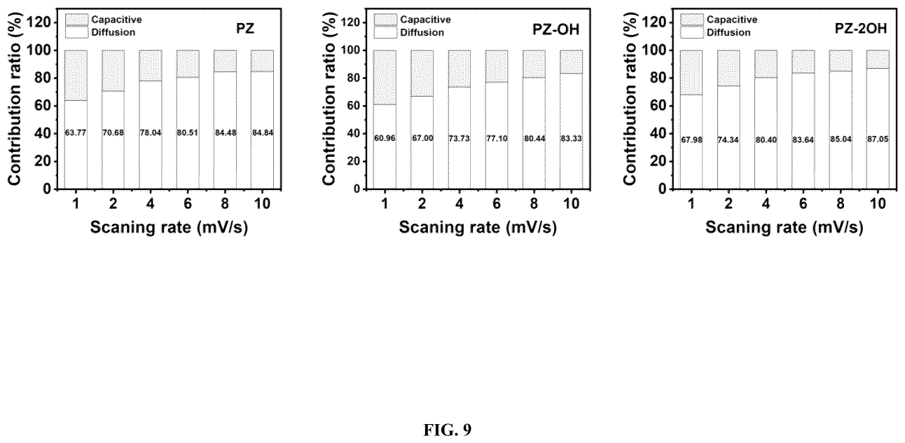

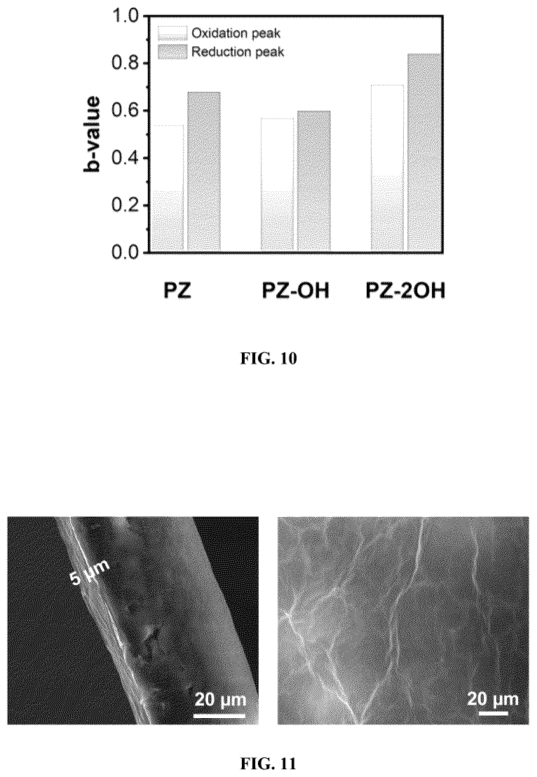

Furthermore, the reaction kinetics, which were investigated through CV tests using scan rates ranging from 1 to 10 mV s⁻¹, also demonstrated the exceptional rate performance of the PZ-2OH anode. The b values obtained from the oxidation and reduction peak currents for PZ, PZ-OH, and PZ-2OH were (0.68, 0.54), (0.60, 0.57), and (0.84, 0.71), respectively, as shown in FIGS. 8-10. These values were determined based on the relationship between scan rate (v) and peak current (i). FIG. 8 showed the CV curves at a scan rate of 1, 2, 4, 6, 8 and 10 mV s⁻¹ and the corresponding log iₚₑₐₖ versus log v of PZ, PZ-OH and PZ-2OH. The upper line represents the reduction peak and the below red line represents the oxidation peak. FIG. 9 showed the capacitive contributions of PZ, PZ-OH and PZ-2OH at different scan rates. FIG. 10 showed the coefficient b of PZ, PZ-OH, and PZ-2OH. (b-value of 0.5 represents a diffusion process, while b-value of 1.0 indicates a capacitive process). The results clearly indicated that the redox processes of the three phenazine derivatives exhibited both ionic diffusion and capacitive behaviors. Notably, the diffusion behavior showed a gradual increase from PZ to PZ-OH, and further to PZ-2OH, as illustrated in FIG. 10.

From the above analysis, it could be found that the ion diffusion kinetics of the phenazine derivatives electrode was affected by the hydroxyl groups, which were available to increase the probability of hydration by forming intramolecular/intermolecular hydrogen bonds, as shown in FIG. 6F. Due to the steric hindrances of adjacent H atoms, PZ exhibits a lower tendency to undergo hydration compared to PZ-OH and PZ-2OH²⁰. The presence of hydrogen bonds facilitates charge transfer, leading to both low ohmic impedance (due to high conductivity) and low charge transfer impedance (due to the ion channel formed by the hydrogen bond network). These combined factors contribute to the excellent high-rate performance of the system.

The interaction of the hydrogen bond was further confirmed through the diagram of the calculated reduced density gradient (RDG) and the corresponding gradient isosurface, as depicted in FIG. 6G. Specifically, the detected green spikes in the sign (λ₂)ρ from −0.02 to 0.00 a.u. proved the existence of weak interactions between PZ-2OH molecules.

### Example 6—Electrochemical Performance of PZ-2OH∥Ni(OH)₂ Battery

The electrochemical performances of the PZ-2OH∥Ni(OH)₂ full cell were assessed using a 1M KOH and 0.1 M LiOH aqueous electrolyte. It was observed that the addition of Li⁺ ions effectively suppressed the O₂ evolution in the anodic nickel-based alkaline battery (ANAB) system.

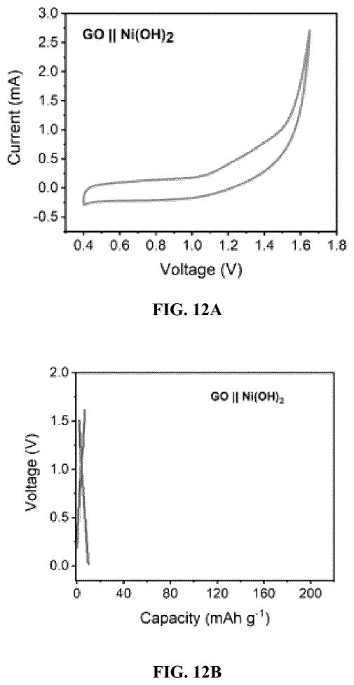

Referring to FIG. 11, a 5 μm thick graphite oxide (GO) membrane was inserted between the anode and the separator. This GO membrane served as a strong shield, effectively preventing anode dissolution. The weight of the film is about 0.67 mg cm⁻². In addition, a GO∥Ni(OH)₂ full cell was also assembled to verify the capacity contribution of the inserted GO membrane. Both CV and GCD images confirmed that the GO membrane exhibited a negligible contribution to the full cell capacity (FIGS. 12A-12B).

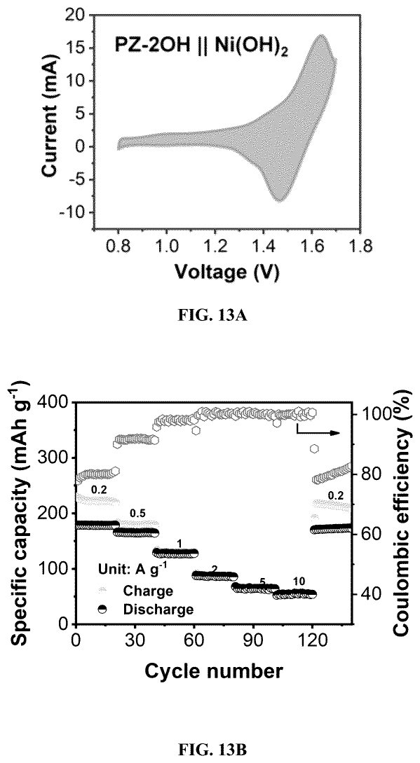

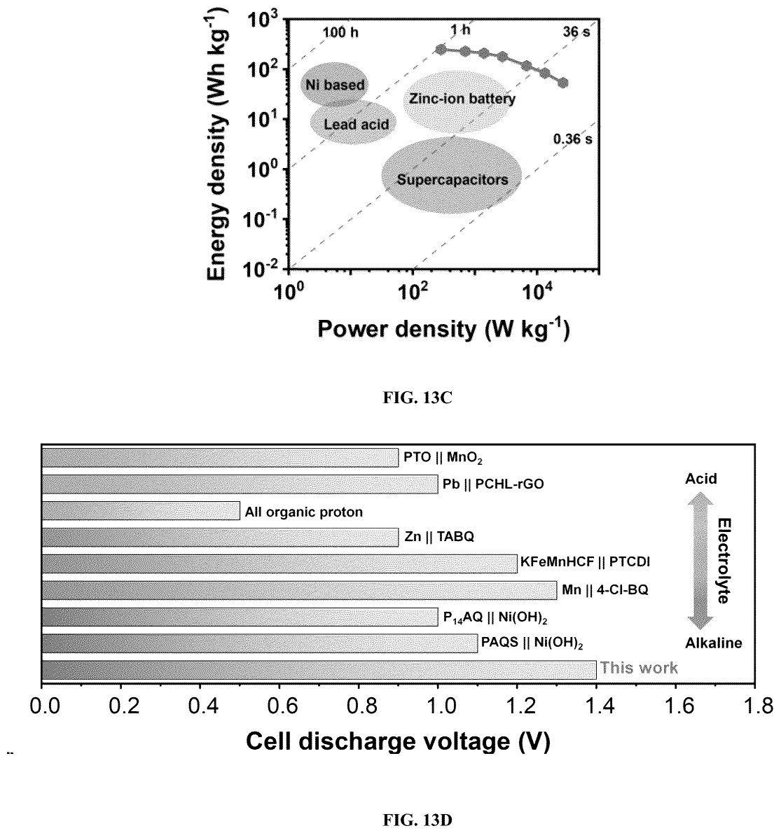

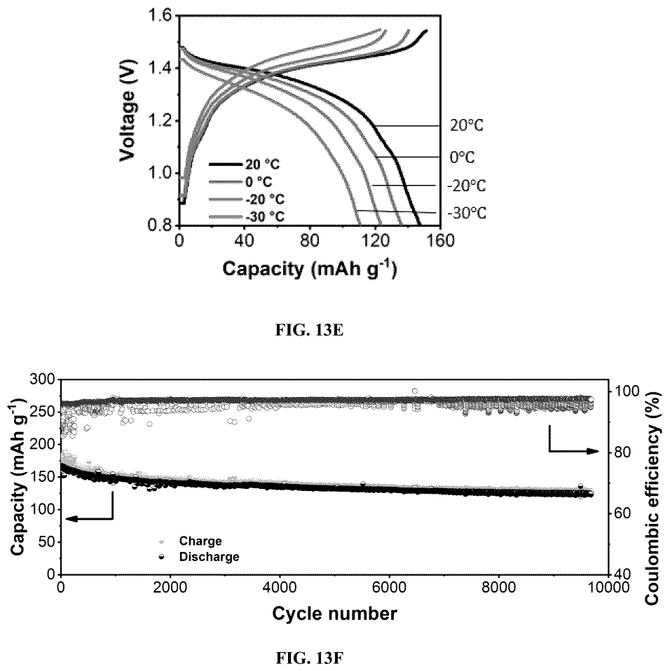

Turning to FIG. 13A, the typical CV curves of the PZ-2OH∥Ni(OH)₂ full cell showed that the oxidation peak at approximately 1.61 V and reduction peak at 1.47 V, which arised from the redox reactions of Ni(OH)₂ cathode and PZ-2OH anode. Such high discharge potential of PZ-2OH∥Ni(OH)₂ batteries were superior to the previously reported Cd∥Ni and MH|Ni batteries^{2, 9}. Moreover, the utilization of a green and cost-effective preparation method provides PZ-2OH with a significant price advantage over other anode materials in alkaline aqueous batteries, as illustrated in FIG. 14.

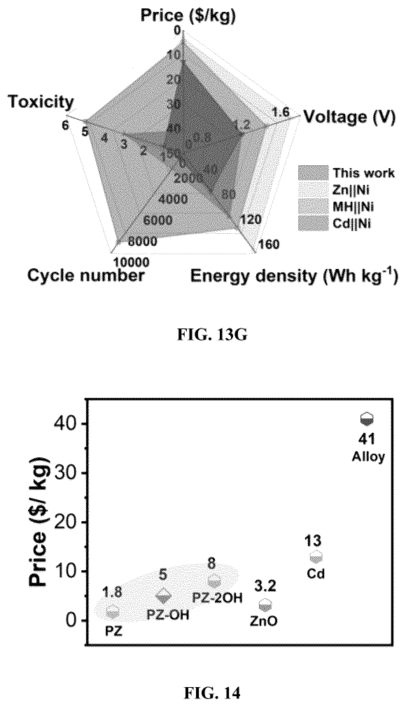

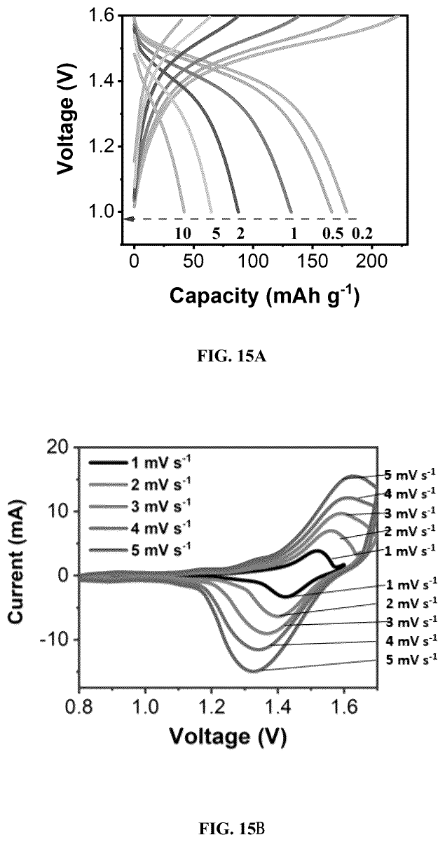

FIG. 13B and FIG. 15A showed the charge/discharge profiles at different current densities (0.2 A g⁻¹, 0.5 A g⁻¹, 1 A g⁻¹, 2 A g⁻¹, 5 A g⁻¹ and 10 A g⁻¹) and rate characteristics of the PZ-2OH∥Ni(OH)₂ batteries. The full cell demonstrated a high discharge capacity (based on the mass of PZ-2OH) of 178 mAh g⁻¹ at a current density of 0.2 A g⁻¹ and a high-rate performance of 64 mAh g⁻¹ at 10 A g⁻¹. In addition, when the current density was restored to 0.2 Ag⁻¹, the discharge capacity is restored to 170 mAh g⁻¹, demonstrating an excellent reversion capability. The high capacity and fast diffusion rate of PZ-2OH anode material provided the PZ-2OH∥Ni(OH)₂ full battery excellent power density (26.2 KW kg⁻¹ at 10 A g⁻¹) and energy density (247 Wh kg⁻¹ at 0.2 A g⁻¹) (FIG. 13C). The high-power density made it close to the supercapacitors with fast charge behavior, while the high energy density was also superior to previously studied lead-acid and nickel-based batteries.

Moreover, in various aqueous electrolytes, the discharge voltage of the full cell was higher than that reported for organic-based batteries, including acid electrolyte (organic pyrene-4,5,9,10-tetraone∥MnO₂ (PTO∥MnO₂)²⁶, Pb∥p-chloranil/reduced graphene oxide (Pb∥PCHL-rGO)²⁷, All organic proton²⁸), mild electrolyte (Zn∥tetraamino-p-benzoquinone (Zn=TABQ)²⁹, KMnHCF∥3,4,9,10-perylenetetracarboxylic diimide (KMnHCF ∥PTCDI)³⁰, Mn∥tetrachloro-1,4-benzoquinone (Mn∥4-Cl-BQ)31), and alkaline electrolyte ((P14AQ∥Ni(OH)₂)³², (PAQS∥Ni(OH)₂)³⁴) (FIG. 13D).

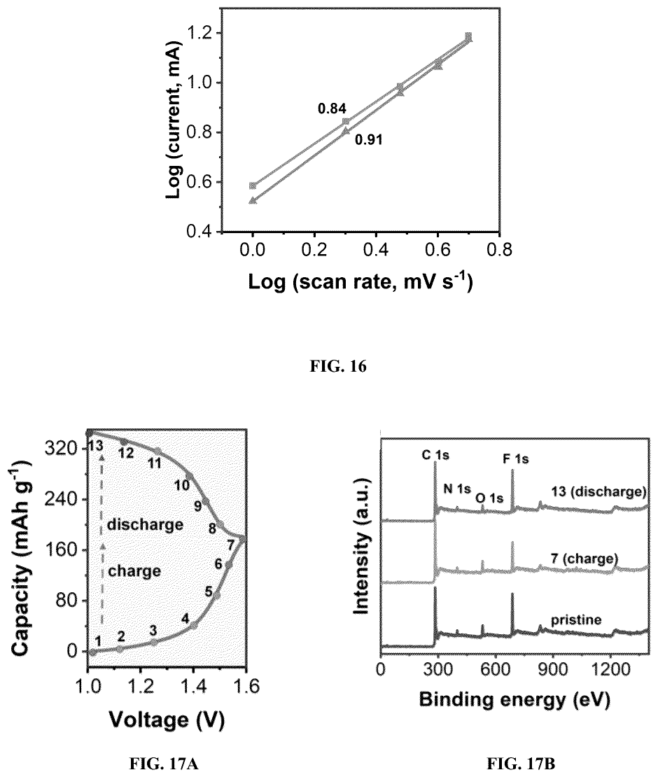

The remarkable rate performance of PZ-2OH∥Ni(OH)₂ batteries was primarily attributed to their fast kinetics, which were investigated through CV tests at various scan rates (FIGS. 15B and 16). The PZ-2OH∥Ni(OH)₂ cell exhibited slopes of 0.84 and 0.91 in the reduction and oxidation processes, respectively. These values indicated the dominance of pseudocapacitive behavior in the cell.

FIG. 13E showed the GCD curves of PZ-2OH∥Ni(OH)₂ full cell at the temperature range of −30° C. to 20° C. and current density of 1 A g⁻¹. Due to the high concentration of alkaline electrolytes and the high electrochemical kinetics of organic electrode, the PZ-2OH∥Ni(OH)₂ batteries could work at −30° C. and maintain 73% capacity.

PZ-2OH∥Ni(OH)₂ batteries were also tested for cycling at 0.5 A g⁻¹, which had a durable cycle performance of more than 9000 cycles, and the average rate of capacitance loss per cycle was approximately 0.075% during the entire cycle process. (FIG. 13F).

FIG. 13G showed comprehensive performance evaluation, including the price, energy density, voltage, toxicity and stability, between the conventional Ni-based batteries (Zn∥Ni, MH∥Ni, Cd∥Ni) and PZ-2OH∥Ni(OH)₂. Therefore, the PZ-2OH∥Ni(OH)₂ had significant advantages over conventional Ni-based batteries. The price was counted according to the anode material; and the nontoxic indicators were graded from 1 to 5, smaller non-toxic data means more toxic. Table 3 was comparison of the comprehensive performances of commercial alkaline nickel-based batteries. The PZ-OH was cheaper than alloy, had lower toxicity than cadmium metal, and had better stability than Zn metal, demonstrating comprehensive performance advantages in price, toxicity, stability, and energy density.

TABLE 3

| | | | | | Energy |
|---|---|---|---|---|---|
| | Price | Cycle | Voltage | | density |
| System | ($/kg) | Numbers | (V) | Nontoxicity | (Wh/kg) |
| This work | 3-10 | 9000 | 1.5 | 5 | 120 |
| Cd∥Ni | 12-14 | 500-2000 | 1.2 | 1 | 60 |
| Metal∥Ni | 40-43 | 500-5000 | 1.2 | 3 | 100 |
| Zn∥Ni | 2.9-3.5 | 300-2000 | 1.7 | 4 | 160 |

Considering three cation species (K⁺, Li⁺ and H⁺) were included in the electrolyte of 1 M KOH+0.1 M LiOH, inductively coupled plasma-optical emission spectrometry (ICP) measurement was performed to identify the charge carrier species in the PZ-2OH anode²¹. From Table 4, although lithium and potassium signals appeared, the content of these elements before and after charge and discharge did not change significantly before and after cycling, indicating that K⁺ and Li⁺ may not be the charge carriers. Detected lithium and potassium signals were probably the residues of the electrolyte on the electrode material. Therefore, the charge carrier in the PZ-2OH anode is more likely to be H^{+ 20, 22, 33, 34}.

TABLE 4

| State | Li (mg/kg) | K(mg/kg) |
|---|---|---|
| pristine | 1.163 | 6.405 |
| charged | 106.4 | 1500.4 |
| discharged | 89.23 | 1704.35 |

To explore the charge storage mechanism, ex-situ XPS and in-situ Raman tests were used to analyze the structural changes of the PZ-2OH electrode in the labeled states during charge-discharge. X-ray photoelectron spectroscopy (XPS; ESCALAB 250) was performed to analyze surface compositions. Raman spectroscopy was conducted on a WITec alpha300 access equipped with a laser of 532 nm to record the changes in chemical bonds during charge/discharge reactions. The content of metal ions (lithium and potassium) was determined by Inductively Coupled Plasma-Optical Emission Spectrometry (ICP-MS: Agilent 7700s ICP-OES: Agilent 720).

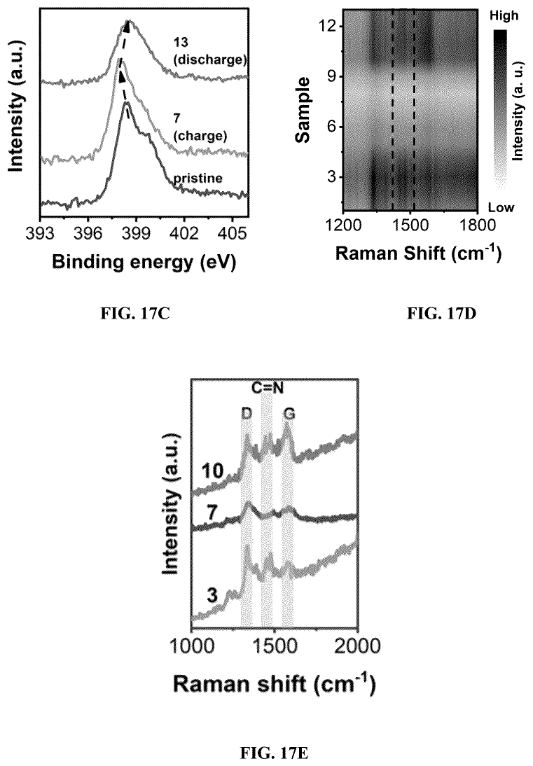

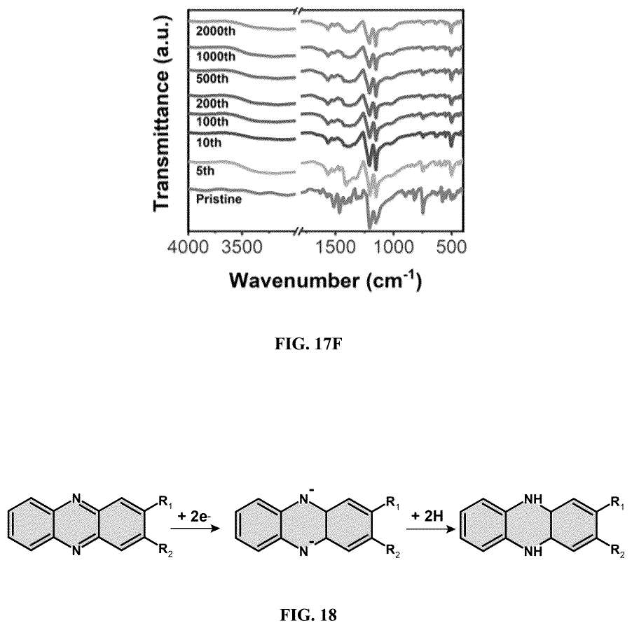

Referring to FIG. 17A, Ex-situ XPS spectra revealed the ion coordination/de-coordination mechanisms of the PZ-2OH anode at the pristine, full charge, and full discharge states. In the XPS spectra of the electrode in the charged/discharge state, no signals from the sodium and lithium elements were observed as shown in FIG. 17B, which further proved that the coordination ions were H⁺. In the high-resolution N 1s spectrum, a noticeable shift of the binding energy of the N peak towards the low-energy region was observed upon charging to 1.6 V. Subsequently, after discharge, the binding energy returned to the high-energy region. This observation suggested that the —C═N bond gained electrons and underwent reduction to form a —C—N bond during the charging process, as depicted in FIG. 17C. Conversely, during discharge, the —C—N bond lost electrons and was oxidized back to the C═N bond, as indicated by previous studies²². These findings aligned with the observations from in-situ Raman spectra (FIG. 17D). Additionally, the characteristic shift of the Raman peak corresponding to the C═N bond further supported this behavior, as illustrated in FIG. 17E. The absorption peak at 1678 cm⁻¹, which corresponded to C═N stretching¹⁷, significantly weakened and eventually disappeared after the battery was charged to 1.6 V. A reverse evolution was observed when the battery was discharged to 0.8 V, indicating that the coordination/in-coordination behaviors exhibited high reversibility.

By combining the results from EDX, ex-situ XPS, and the in-situ Raman tests characterization, the charge/discharge storage behavior of PZ-2OH was based on the n-type doping reaction, as depicted in FIG. 18. During the initial charging process, the PZ-2OH anode underwent reduction by accepting delocalized electrons, forming a negative radical. Simultaneously, H⁺ ions were inserted into the PZ-2OH to maintain electroneutrality. In the subsequent charging process, the negatively charged PZ-2OH anode was then reduced back to its neutral state by releasing the electrons.

In addition, the reduced states of the PZ-2OH anode were also monitored by ex-situ FTIR (FIG. 17F) after different cycles. No obvious chemical decomposition was detected except the slight changes for the initial few cycles, indicating good chemical stability in strong alkaline solutions, which ensured the low capacity-fade rates during the long cycling.

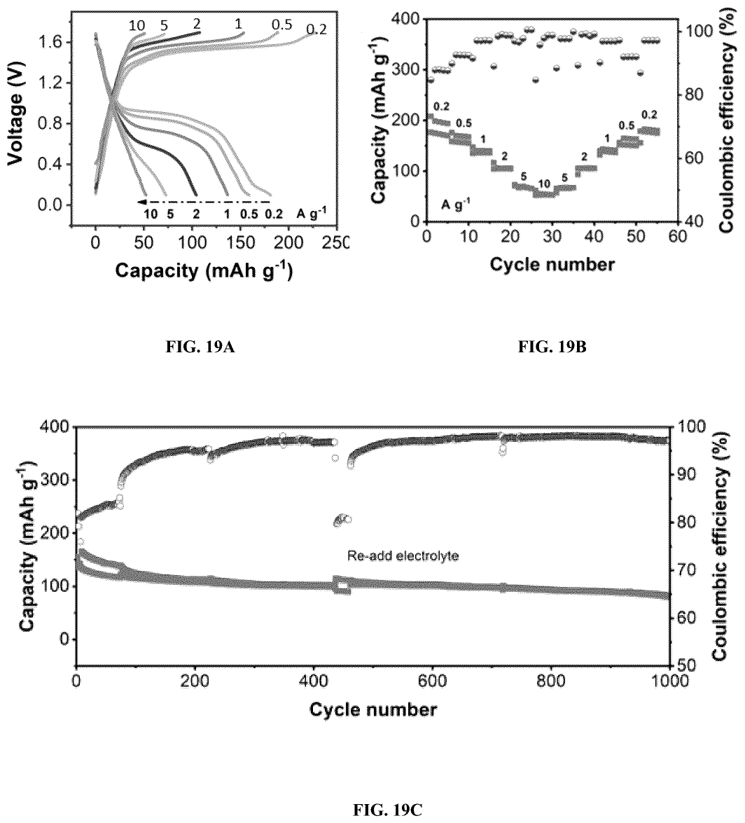

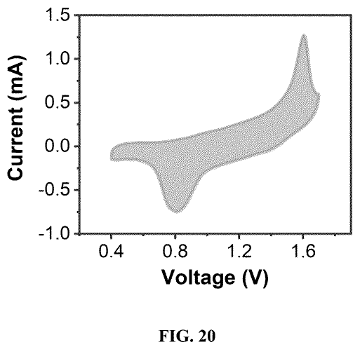

To further verify the adaptability of PZ-2OH under alkaline conditions, the PZ-2OH∥Air full battery was assembled. The GCD curves and rate capabilities of the PZ-2OH∥Air battery were shown in the FIGS. 19A and 19B, respectively. Turning to FIG. 20, the CV curves of PZ-2OH∥Air full cell showed typical oxidation and reduction peaks at 0.8 and 1.5 V, which confirmed the possibility of PZ-2OH being used as an anode for air cells under alkaline conditions. The cell exhibited specific capacities of 181.3, 158.9, 136.7. 104.2, 72.9, and 52.3 mAh g⁻¹ at 0.2. 0.5, 1, 2, 5, and 10 A g⁻¹ based on the PZ-2OH mass, indicating the excellent rate performance. It is noteworthy that the rate performance observed in the PZ-2OH∥Air full cell was relatively lower compared to that in the PZ-2OH half battery (FIG. 6A). This disparity can be attributed to the limited catalytic activity of the air cathode under high current conditions.

Moreover, the cycle performance of PZ-2OH∥Air full cell showed a high capacity retention of approximately 65.8% in the initial 400 cycles, as demonstrated in FIG. 19C. A noticeable drop in capacity occurred as the cycle continued, which was caused by electrolyte loss in the open system. After the electrolyte was refilled into the battery, the capacity was immediately restored, suggesting that the recession in capacity was mainly related to the loss of electrolyte and decreased of ion transportation. The capacity of the full cell remained 80.4 mAh g⁻¹ after 1000 cycles. The obtained results provide compelling evidence that the developed PZ-2OH material functions effectively in alkaline electrolytes. This material demonstrates great potential for application in various alkaline batteries, enabling them to deliver superior energy/power density and rate capacity.

In summary, the present invention presents a one-step, efficient, environmentally friendly, and cost-effective preparation process for a series of phenazine derivatives. These derivatives are synthesized by utilizing benzoquinone derivatives and phenylenediamine derivatives as reaction precursors, with ethanol and/or water as the dispersion medium. Three phenazine derivatives, phenazine (PZ), 2-hydroxyphenazine (PZ-OH) and 1,2-dihydroxyphenazine (PZ-2OH), which contain varying numbers of hydroxyl groups. They are used as model compounds to investigate the influence of grafted functional groups on the electrochemical redox properties of phenazine as an anode material in alkaline-based batteries. The cathode materials employed are commercial nickel hydroxide (Ni(OH)₂) for alkaline nickel-based batteries and Pt/C for alkaline air batteries. The optimized PZ-2OH∥Ni(OH)₂ batteries can exhibit a high capacity of 208 mAh g⁻¹ (anode), a high energy density of 247 Wh kg⁻¹ (anode), high power density of 26.2 KW kg⁻¹ at 10 A g⁻¹, and exceptional cycling stability with over 9000 cycles and a low capacity decay rate of approximately 0.075% per cycle.

### Definitions

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they allow for elements not explicitly recited, but exclude elements that are found in the prior art or that affect a basic or novel characteristic of the present invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

References in the specification to “one embodiment”, “an embodiment”, “an example embodiment”, etc., indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

Other definitions for selected terms used herein may be found within the detailed description of the present invention and apply throughout. Unless otherwise defined, all other technical terms used herein have the same meaning as commonly understood to one of ordinary skill in the art to which the present invention belongs.

It will be appreciated by those skilled in the art, in view of these teachings, that alternative embodiments may be implemented without undue experimentation or deviation from the spirit or scope of the invention, as set forth in the appended claims. This invention is to be limited only by the following claims, which include all such embodiments and modifications when viewed in conjunction with the above specification and accompanying drawings.

### INDUSTRIAL APPLICABILITY

The market for electrode materials is projected to grow from USD 25.9 billion in 2022 to USD 52.6 billion by 2027, according to a new market research report by MarketsandMarkets™. In comparison to traditional inorganic materials, organic materials have emerged as the preferred choice due to their environmental friendliness, sustainability, flexible structure designability, and abundance in the Earth's crust. The structural diversity of organic materials also allows for battery modification and innovation.

In the present invention, a series of phenazine derivatives with varying types and amounts of side substituents were prepared using a simple and environmentally friendly condensation method. By adjusting the electron donating/withdrawing ability of these side groups, it is possible to customize the organic anode to achieve desired electrochemical performances. The developed organic phenazine derivatives have broad applications in alkaline battery systems, including alkaline nickel-based batteries and alkaline air batteries. These advancements greatly contribute to the development of alkaline aqueous batteries. The developed organic phenazine materials exhibit ultralow redox potentials and extremely high cycling stability. Their low cost, simple preparation method, environmentally friendly characteristics, and excellent electrochemical performance make them highly promising for large-scale stationary energy storage, portable high-energy devices, and high-safety energy storage systems.

### REFERENCES: THE DISCLOSURES OF THE FOLLOWING REFERENCES ARE INCORPORATED BY REFERENCE

1. J. F. Parker, C. N. Chervin, I. R. Pala, M. Machler, M. F. Burz, J. W. Long and D. R. Rolison, Science, 2017, 356, 415-418.
2. T. Sakai, I. Uehara and H. Ishikawa, J. Alloy Comp., 1999, 293, 762-769.
3. T. J. Sun, C. Liu, J. Y. Wang, Q. S. Nian, Y. Z. Feng, Y. Zhang, Z. L. Tao and J. Chen, Nano Res., 2020, 13, 676-683.
4. W. Zhou, D. Zhu, J. He, J. Li, H. Chen, Y. Chen and D. Chao, Energy Environ. Sci., 2020, 13, 4157-4167.
5. F. Wang, J. Xie, D. Zheng, F. Yang, H. Zhang and X. Lu, Adv. Mater., 2022, 34, 2200085.
6. L. Jiang, D. Dong and Y.-C. Lu, Nano Res. Energy, 2022, 1, e9120003.
7. S. Chen, T. Wang, L. Ma, B. Zhou, J. Wu, D. Zhu, Y. Y. Li, J. Fan and C. Zhi, Chem, 2022.
8. M. J. D'Ambrose, D. E. Turney, G. G. Yadav, M. Nyce and S. Banerjee, ACS Appl. Energy Mater., 2021, 4, 3381-3392.
9. F. Feng, M. Geng and D. Northwood, Int. J. Hydrogen Energy, 2001, 26, 725-734.
10. S. Chen, C. Peng, D. Xue, L. Ma and C. Zhi, Angew. Chem., 2022, 134, e202212767.
11. J. S. Ko, A. B. Geltmacher, B. J. Hopkins, D. R. Rolison, J. W. Long and J. F. Parker, ACS Appl. Energy Materials, 2018, 2, 212-216.
12. S. Yang, H. Lv, Y. Wang, X. Guo, L. Zhao, H. Li and C. Zhi, Angew. Chem. Int. Ed., 2022, 61, e202209794.
13. J. Kim, S. Ko, C. Noh, H. Kim, S. Lee, D. Kim, H. Park, G. Kwon, G. Son and J. W. Ko, Angew. Chem., 2019, 131, 16920-16925.
14. G. Liang, X. Li, Y. Wang, S. Yang, Z. Huang, Q. Yang, D. Wang, B. Dong, M. Zhu and C. Zhi, Nano Res. Energy, 2022, 1, e9120002.
15. C. Luo, O. Borodin, X. Ji, S. Hou, K. J. Gaskell, X. Fan, J. Chen, T. Deng, R. Wang and J. Jiang, Proc. Nat. Acad. Sci., 2018, 115, 2004-2009.
16. H. Cui, L. Ma, Z. Huang, Z. Chen and C. Zhi, SmartMat, 2022.
17. S. Zhang, S. Long, H. Li and Q. Xu, Chem. Eng. J., 2020, 400, 125898.
18. Q. Wang, Y. Liu and P. Chen, J. Power Sources, 2020, 468, 228401.
19. G. Dai, Y. Liu, Z. Niu, P. He, Y. Zhao, X. Zhang and H. Zhou, Matter, 2019, 1, 945-958.
20. A. Hollas, X. Wei, V. Murugesan, Z. Nie, B. Li, D. Reed, J. Liu, V. Sprenkle and W. Wang, Nat. Energy, 2018, 3, 508-514.
21. C. Zhang, W. Ma, C. Han, L.-W. Luo, A. Daniyar, S. Xiang, X. Wu, X. Ji and J.-X. Jiang, Energy Environ. Sci., 2021, 14, 462-472.
22. Z. Tie, L. Liu, S. Deng, D. Zhao and Z. Niu, Angew. Chem., 2020, 132, 4950-4954.
23. L. Ma, S. Chen, D. Wang, Q. Yang, F. Mo, G. Liang, N. Li, H. Zhang, J. A. Zapien and C. Zhi, Adv. Energy Mater., 2019, 9, 1803046.
24. K. C. Kim, T. Liu, S. W. Lee and S. S. Jang, J. Am. Chem. Soc., 2016, 138, 2374-2382.
25. T. Liu, K. C. Kim, B. Lee, Z. Chen, S. Noda, S. S. Jang and S. W. Lee, Energy Environ. Sci., 2017, 10, 205-215.
26. Z. Guo, J. Huang, X. Dong, Y. Xia, L. Yan, Z. Wang and Y. Wang, Nat. Commun., 2020, 11, 1-9.
27. F. Yue, Z. Tie, S. Deng, S. Wang, M. Yang and Z. Niu, Angew. Chem. Int. Ed., 2021, 60, 13882-13886.
28. R. Emanuelsson, M. Sterby, M. Strømme and M. Sjödin, J. Am. Chem. Soc., 2017, 139, 4828-4834.
29. Z. Lin, H.-Y. Shi, L. Lin, X. Yang, W. Wu and X. Sun, Nat. Commun., 2021, 12, 1-9.
30. L. Jiang, Y. Lu, C. Zhao, L. Liu, J. Zhang, Q. Zhang, X. Shen, J. Zhao, X. Yu and H. Li, Nat. Energy, 2019, 4, 495-503.
31. S. Bi, S. Wang, F. Yue, Z. Tie and Z. Niu, Nat. Commun., 2021, 12, 6991.
32. C. Liu, T. Ma, K. Xia, X. Hou, Q. Nian, Y. Cai and J. Liang, Sustain. Energy Fuels, 2020, 4, 132-137.
33. J. Xu, S. Pang, X. Wang, P. Wang and Y. Ji, Joule, 2021, 5, 2437-2449.
34. S. Pang, X. Wang, P. Wang and Y. Ji, Angew. Chem. Int. Ed., 2021, 60, 5289-5298.

What is claimed is:

1. A phenazine derivative-based alkaline battery, comprising:
an anode formed by a phenazine derivative having 1 to 4 side group substituents;
a cathode;
a separator placed between the cathode and the anode; and
an electrolyte disposed in a space between the cathode and the anode,
wherein the phenazine derivative-based alkaline battery exhibits a reversible capacity of at least 170 mAh g⁻¹ at 0.2 A g⁻¹, a power density of at least 20 KW kg⁻¹ at 10 A g⁻¹, and a stable cyclability over 9000 cycles.

2. The phenazine derivative-based alkaline battery of claim 1, wherein the side group substituents comprise electron-donating groups or electron-withdrawing groups.

3. The phenazine derivative-based alkaline battery of claim 2, wherein the electron-donating groups comprise amino, hydroxyl, or a combination thereof, or the electron-withdrawing groups comprise methyl, carboxyl group, or a combination thereof.

4. The phenazine derivative-based alkaline battery of claim 1, wherein the anode and cathode comprise a current collector, at least one active material, one or more electronic conductive particles, and at least one binder.

5. The phenazine derivative-based alkaline battery of claim 4, wherein the current collector comprises carbon nanotube paper, carbon cloth, and nickel foil.

6. The phenazine derivative-based alkaline battery of claim 4, wherein the at least one active material comprises phenazine (PZ), 2-hydroxyphenazine (PZ-OH), and 1,2-dihydroxyphenazine (PZ-2OH).

7. The phenazine derivative-based alkaline battery of claim 4, wherein the one or more electronic conductive particles comprise carbon nanotubes, ketjenblack, and super P.

8. The phenazine derivative-based alkaline battery of claim 4, wherein the at least one binder comprises sodium carboxymethylcellulose (CMC-Na), polyvinylidene difluoride (PVDF), and Polytetrafluoroetylene (PTFE).

9. The phenazine derivative-based alkaline battery of claim 1, wherein the cathode comprises Ni(OH)₂ and Pt/C.

10. The phenazine derivative-based alkaline battery of claim 1, wherein the electrolyte comprises a solvent and a solute, and the solvent comprises deionized water, the solute comprises sodium hydroxide, potassium hydroxide, and lithium hydroxide.

11. The phenazine derivative-based alkaline battery of claim 10, wherein the concentration of the solute is in a range of 1 M to 6 M.

12. The phenazine derivative-based alkaline battery of claim 1, wherein the phenazine derivative-based alkaline battery further comprises a graphene oxide (GO) film, wherein the phenazine derivative-based alkaline battery is assembled by inserting the GO film between the anode and the separator, and the electrolyte is added between the anode and the cathode.

13. The phenazine derivative-based alkaline battery of claim 1, wherein the phenazine derivative-based alkaline battery retains at least 10% of the initial capacity, and the discharge potential of the phenazine derivative-based alkaline battery is lower than −0.8 V.

14. The phenazine derivative-based alkaline battery of claim 1, an increase in the number of the side group substituents results in a reduction in electron affinity and a decrease in the redox potential.

15. A method for preparing the phenazine derivative of claim 1, comprising:
preparing at least one precursor comprising at least one benzene derivative, at least one phenylenediamine derivative, and a solvent;
dissolving the at least one benzene derivative into the solvent to form a uniform solution A;
mixing the at least one phenylenediamine derivative with the solution A to form a suspension; and
filtering and washing the suspension with deionized water until a filtrate becomes colorless,
wherein the at least one benzene derivative and the at least one phenylenediamine derivative has a molar ratio in a range of 1:1-1:0.8.

16. The method of claim 15, wherein the method further comprises concentrating the mixture at reduced pressure and adding deionized water to obtain a suspension before filtering and washing the suspension.

17. The method of claim 15, wherein the at least one benzene derivative comprises benzoquinone or 2,5-dihydroxy-1,4-benzoquinone.

18. The method of claim 15, wherein the at least one phenylenediamine derivative has the following chemical structure: 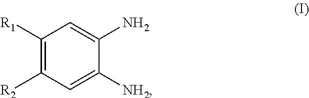
wherein the R₁ and R₂ are independently selected from H, Cl, COOH, or CH₃.

19. The method of claim 15, wherein the solvent comprises ethanol, deionized water, or a combination thereof.

20. The method of claim 15, wherein the step of mixing the at least one phenylenediamine derivative with the solution A to form a suspension has a reaction temperature ranges from −10° C. to 100° C., and a reaction time ranges from 5-10 hours.
